(19) United States
(12) Patent Application Publication
(10) Pub. No.: US 2011/0311634 A1
(43) Pub. Date: Dec. 22, 2011

(54) FORMULATIONS FOR ANIMAL FEED COMPRISING BUTYRATE SALT

(75) Inventor: Luc Goethals, Gijzenzele (BE)

(73) Assignee: SANLUC INTERNATIONAL NV, GIJZENZELE (BE)

(21) Appl. No.: 13/133,666

(22) PCT Filed: Dec. 7, 2009
(86) PCT No.: PCT/EP2009/008720

§ 371 Date: Sep. 5, 2011

(30) Foreign Application Priority Data
Dec. 9, 2008 (GB) 0822398.4

(51) Int. Cl. A61K 31/19; A61K 36/00; A61K 36/06; A61P 39/00; A61K 36/49; A61P 3/00; A61P 31/00; A61K 9/50; A61K 31/20

(52) U.S. Cl. 424/490; 514/557; 424/725; 424/195.16; 514/558; 424/769

(57) ABSTRACT

The present invention relates to formulations for animal feed comprising coated granules comprising butyrate salt; the butyrate salt is preferably the salt of sodium or calcium salt. The coated granules have a particle size of 0.1 mm or more, preferably 0.2 mm or more and about 2 mm or less, and preferably about 1 mm or less. The coated granules comprise a binder and a coating. Further, the butyrate salt can be combined with another active ingredient which may be chosen from the group consisting of plant extracts, prebiotic compounds, probiotics, yeast extracts, middle-length chain fatty acids, unsaturated long chain fatty acids, lactate salt, fat soluble vitamin, and toxin absorbing compound.

The invention relates to formulations for animal feed comprising butyrate salt.

Butyrate salt is a known feed additive (raw material) to increase the health of farm animals such as poultry, swine and cattle. Generally, the additive is used as sodium or calcium salt, although other cations can be used. Butyrate has a positive effect on a number of characteristics of the animals, however, it is a product which smells, and can be unpleasant to handle, thereby limiting its actual use.

It is an object of the invention, to provide formulations for animal feed comprising butyrate salt with enhanced beneficial effects.

It is a further object of the invention, to provide butyrate salt comprising formulations with better handling characteristics and less or no odor.

The invention thus provides for formulations comprising butyrate salt in granular form with other active ingredients.

The invention furthermore provides for granules comprising butyrate salt, the granules having low odor.

The invention furthermore provides for formulations for animal feed comprising granules of butyrate salts, preferably coated granules, wherein the formulation further comprises other active ingredients.

### Butyrate Salt

The butyrate salt can be a metal salt or organic salt such as an ammonium salt. The salt may be liquid, (and used on a carrier) but preferably is a solid at a temperature below 70° C. Suitable cations include, but are not limited to calcium, sodium, potassium, zinc, manganese, copper, selenium, ammonium and the like, and mixtures thereof. Potassium or ammonium salts are liquid at room temperature, and can be used when absorbed on a carrier. Preferably, the cation is chosen because of stability of the salt, and because of nutritional value. Preferable, the salt is a sodium or calcium salt, and most preferably a calcium salt, as that salt is less hygroscopic and more stable. Other suitable cations are selenium, potassium, magnesium, copper, and/or zinc. The latter metal ions can be used in admixture with—for example—calcium as to supply the feed with organic metal compounds which are useful for the animal.

The butyrate salt generally is made from butyric acid and a base. Suitable butyric acid can be from chemical source, or from fermentative (biological) source. Suitable bases include, but are not limited to calciumhydroxide, calciumoxide, sodiumhydroxide, ammonium hydroxide, zinc oxide or hydroxide, copper oxide or hydroxides, manganese oxide or hydroxide and selenium oxide. Water that is formed during the reaction needs to be removed, and preferably is evaporated. As the reaction is exothermic, the evaporation can be effected with little or no external energy input. Otherwise, filtering crystallized material is possible as well. A suitable process is described in U.S. Pat. No. 1951250, EP 630579 and US 2004/010040.

The butyrate salt is generally amorphous or multi-crystalline or mixtures hereof.

The size of the butyrate size particles preferably is such that they can be handled easily. Generally, the particle size is about 1 μm or more, preferably 0.01 mm or more and more preferably 0.1 mm or more. Generally, the particle size is 2 mm or less, preferably 1 mm or less. Suitable particle sizes include 0.1, 0.4-0.6, 0.5 mm and 0.7 mm.

The butyrate preferably is used as a coated granular product. This has the advantage of less or virtually no odor, and improved handling characteristics. The coated granules preferably have a particle size of 0.05 mm or more, and more preferably 0.1 mm or more, and most preferably 0.2 mm or more. Generally, the coated particles will be about 2 mm or less, preferably 1 mm or less, and most preferable about 0.8 mm or less.

The particle size is measured with classical sieve analysis. The high end value is an absolute value (i.e. all particles can pass through a sieve to give particles of e.g. less than about 1 mm). The lower end value is given as the size where more than 95 wt % is larger than the value given, preferably more than 98% and most preferably more than 99% wt is larger than the value stated as lower end value. Attrition may cause some amount of smaller particles to become present, for example during transportation.

In a preferred embodiment of the invention, the butyrate salts is comprised in granular particles that are coated. Such granular particles preferably comprise a binder and a coating. The binder functions as a matrix to glue the butyrate salt particles together in order to obtain a strong granule and the coating gives increased strength and further reduces odor. The binder and coating may be the same or different, and can be chosen from a wide variety of materials. It is preferred to use a compound that is present in normal feed compositions, such as fatty acid derivative, carbohydrate or protein. It is particularly preferred to use a fatty acid derivative (shortly noted as fat) as a coating because fat increases the flowability of the product. Fat is also preferred as binder because it further aids in protection against moisture. Suitable other materials include starch, dextrine, cellulose, cellulose and derivatives thereof, such as carboxymethylcellulose (cmc).

The granular particles preferably comprise a core, where the majority of the butyrate salt is present, and a coating, where the concentration of the butyrate salt is about 20 wt % or less. Preferably, the amount of butyrate salt in the coating of the particles is about 10 wt % or less and even more preferably about 5wt % or less. The amount may be as low as 2-3 wt %.

The butyrate is considered to exert its activity in the intestine, and in particular the small (but also large) intestine. Hence, another advantage of using fat is, that this aids in reaching the intestine. However, other binders and coatings can be used as well for such purpose; for example carboxymethylcellusose is able to reach the large intestine.

Preferably, the granule is stable at about 50° C. or higher for improved storage stability. In a more preferred embodiment, the granules are stable up to about 55° C. or higher and even more preferably up to about 60° C. or higher, such that the granules withstand the temperature at which calf-milk is prepared (about 56-60° C.). The upper limit of the stability is not critical, but generally, at a temperature of about 150° C., the product will degrade, and a stability temperature of about 100° C. or less will be sufficient in most cases. Stable means in this context, that the granules remain integral particles if kept for 10 min in water at the test temperature. Stability at elevated temperature is also important to withstand temperatures during extrusion and/or pelletizing. In pelletizing feed products often temperatures of up to 90° C. are reached for several seconds. A heat stability of 10 min in water at for example 60° C. is generally also sufficient to withstand 90° C. for a few seconds.

Suitable fats are glyceride esters of fatty acids, alkyl esters of fatty acids, fatty acids, hydroxyl fatty acid analogues of the above, fatty alcohols (like waxes) and mixtures thereof. Fatty acids generally have a number of carbon atoms of 12 or higher, preferably of 16 or higher such as for example 18, 20 and/or 22 carbon atoms. Suitable fatty acids include hydroxy-fatty acids. Preferably, the fatty acid derivative is a predominantly triglyceride ester of fatty acids, and more preferably is the harder fraction of vegetable oils. Preferably, the fatty acids or fatty acid esters have a melting point of about 50° C. or higher, preferably 57° C. or higher. Generally, the melting point will be about 120° C. or lower, preferably 100° C. or lower.

In a preferred embodiment, glycerol fatty acid esters (or fats) are used, as these are well available and are stable. Suitable fats include fats from vegetables or animals. Fats from vegetables are preferred because of hygienic reasons. Suitable examples of fats include the hard fractions of coconut oil, palm oil and the like. The fats can be partly or fully hydrogenated oils or (semi)purified oils like hydrogenated castor oil and the like.

Granules can be prepared for example in a rotary granulator, preferably adding the butyrate salt and the binder to build granules. Thereafter, the coating can be sprayed on the granules, preferably also in the rotary granulator, but this can also be performed in a fluid bed.

Preferably, the amount of binder and coating is about 80 wt % or less. Such an amount of binder and coating is suitable if a liquid or hygroscopic butyrate salt is used. In a preferred embodiment, the amount of binder and coating is about 50 wt % or less, even more preferably about 35 wt % or less, and in certain embodiments about 25 wt % or less. More binder and coating generally increases the strength of the granules, and further lowers the odor. However, because the amount of butyrate salt is also lowered, the costs per feed additive value increases, which is a disadvantage. Generally, the amount of binder and coating will be about 5 wt % or more, and preferably about 10 wt % or more, in order to have improved strength and less odor.

Generally, the granules contain about 40 wt % butyrate salt or more, preferably about 50 wt % or more. Preferably, grades are available with about 60 wt % butyrate or more, such as for example about 70 wt %.

Next to the binder, coating and butyrate salt, the granules may contain other components such as excipients, fillers or other active materials. Examples of further materials include: aromatic compounds to exert a more attractive odor; emulgating compounds and/or polymers to increase viscosity and/or hardness of the coating, such as for example acrylates, polyvinylpyrrolidone polymers and the like; antioxidants; coloring agents and the like.

In a preferred embodiment of the invention, the granules contain a butyrate salt as the major biologically active component in the granules. Biologically active is used herein to denote a health or nutrition effect, and not only the energy value of for example fat or carbohydrates. Alternatively, it is possible to add further biologically active compounds during granulation and/or coating of the granules, to produce granules with two or more active ingredients.

Generally, butyrate, preferably as calcium butyrate, is present in the animal feed in an amount of 10 g per ton or more, preferably about 50 g per ton or more. Generally, the amount will be about 3 kg per ton or less, preferably about 1 kg per ton or less and even more preferred about 500 g or less. The upper limit is not critical, but an increased amount would add costs with little or no benefit. The lower level amount may be important to achieve the desired effect, and may take some experimentation to find optimal amounts from economic perspective.

For example, in case of the use of encapsulated calcium butyrate with 70% active butyrate available, an amount of about 100 g or more is preferred per ton of feed. Generally an amount of 3 kg or less per ton of feed is preferred.

### Other Ingredients in the Formulation

The feed additive formulation of the present invention comprises preferably the butyrate salt and at least one further active ingredient, preferably a further biologically active compound. Suitable further biological effects are bacterial infection inhibition, coloring, growth, quality of the food produced, such as protein value and the like.

Generally, the combination of two active compounds has some benefit over the single compound only, but, generally, the effect is less than an additive effect. In the present invention, preferably, the at least two compounds have an additive, or even more preferably a synergistic effect together with the butyrate salt. The combined effect can be defined as synergistic (reinforcing) and/or additive (broadening), without having an antagonistic or diminishing effect on the activity of other additive(s). It was unexpected that in particular coated granulated butyrate shows generally an additive or synergistic effect in stead of a less-than-additive effect as most of the other feed additives do.

In the formulation comprising a butyrate salt and at least one other active ingredient, the butyrate salt is preferably a salt as described above. More preferably, butyrate salts are used as coated granular particles in the formulation with another active ingredient. The use of butyrate salt as coated granular particles has the advantage that the butyrate salt does have very little or no interaction (or reaction) with the other ingredient in the feed additive formulation. This is preferred because butyrate salt may be hygroscopic or still has some free butyric acid that is able to react with other ingredients. The use of properly coated butyrate salts obviates these problems.

Preferably, the further active compound is a plant extract, short and/or medium chain fatty acid(s), lactic acid or derivate, probiotic(s), prebiotic(s), yeast extract(s) or derivative(s), nucleotides, toxin absorbing clay, long chain multiple-unsaturated fatty acid(s), fat soluble vitamins or mixtures or combinations of these.

The additive preferably has a health effect in that the need for antibiotics is reduced. In another embodiment, the additive has a nutritional value in that the additive improves the use of the feed.

The formulation with additive is preferably aimed to exert its main effect in the small intestine. Hence, the formulation is designed such that a large part passes the stomach and that a large part is digested in the small intestine.

A suitable solubility test may be used in analogy to the test described in S. Boisen, and J Fernandez in Animal Feed Science Technology 68 (1997) 277-286. The amount dissolved in a stomach type fluid can be determined as follows: about 2 g of coated butyrate formulation is (without grinding) put in about 105 ml solvent at pH 2 as described in the reference with 3 ml of pepsin solution containing 75 mg pepsin (porcine, 2000 FIP-U/g). It is preferred that the amount dissolved in 1 hr preferably is less than 20 wt %, preferably less than 10 wt % at 39° C. Even more preferable, less than 20% preferably less than 10% dissolved formulation is measured after 2 hr at 39° C. The amount dissolved in the intestine can thereafter be determined as follows: A solution obtained after 1 hr according step 1 is thereafter brought to pH 6.8, as described in the reference, and 3 ml of pancreatin solution containing 300 mg pancreatin (porcine, grade IV, Sigma) is added, and the vessel is held at 39° C. while stirring for 2-4 hrs. Preferably, the amount dissolved after 4 hours is about 80% or more, even more preferably about 90% or more of the formulation. Alternatively, preferably the amount dissolved after 2 hr is about 80% or more, and may be 90% or more. The amount dissolved refers to the amount of butyrate.

The feed additive preferably contains about 50 g or more of butyrate salt, about 50 g or more of each of another component, to be added to a ton of feed. It is preferred to formulate the feed additive such that one kg preferably is added to one ton of feed. In such case, it is preferred to have about 10% or more of any of the required components per kg of feed additive. Generally, any of the components will be present in amount of 70% or less. Preferably, respective amounts are about 20% or more of each compound, and about 50% or less of each compound. In formulating such feed additive, it is preferred to first determine the amount of butyrate salt needed, and thereafter determine a suitable amount of a further critical component. Generally, one of the other compounds may be less critical, and can be used to make 1 kg.

It is further feasible to add organic or inorganic filler, like calciumcarbonate, silica, sepiolite, wheat bran, corn cob meal, chicory pulp and the like, to make a total formulation 1 kg.

### Plant Extracts

In one preferred embodiment, a feed additive comprises a (preferably encapsulated) butyrate salt component and a plant extract.

Preferably, the plant extract is an active compound; active having the meaning that a useful biological effect is observed.

Suitable plant extracts include extracts having the effect to improve quality or quantity of the food product (animal or animal products). Suitable effects include preventing intestine disorders, reducing bacterial growth (anti-bacterial effect), reducing mold growth (anti mold effect), improved feed efficiency (higher daily wait gain of animals), improved food product like less urea in milk, less fractures in egg shell, improved coloring, more fat or protein in milk, and the like.

Examples of suitable plant extracts include, but are not limited to oregano oil or its components, thymol and carvacrol, cinnamaldehyde, ionone, artemisinin, eugenol, citrusextract, tannine extracts and grape-extracts.

In a preferred embodiment of the invention, as plant extracts, tannin extract of the sweet chestnut tree is used. Such extract exhibits mold and bacterial growth inhibiting characteristics. Suitable amounts include about 50 g or more per ton of feed, preferably about 100 g or more per ton of feed. Generally, the amount will be about 10 kg per ton of feed or less, preferably about 5 kg per ton or less. Lower amounts may be suitable for poultry, whereas the amounts from the higher range end are suitable in rabbit, and the middle range for cattle and swine.

The combined use of butyrate salt and tannin exhibits a synergistic effect on growth and health of animal.

In another embodiment, the plant extracts comprise enhanced amounts of polyphenols like flavones, flavonones, anthocyanins and catechins. Suitable amounts may depend on the level of purity of the plant extracts, and if very pure may be as little as 10 g per kg feed additive for 1 tonne of feed, but generally, an amount of about 50 g or more as described above will be useful.

Simple additives that for example only limit ammonia excretion are less preferred, and are preferably not used.

### Toxin Absorbing Compound

In a preferred embodiment, a toxin absorbing compound is present in the feed additive according to the present invention. The toxin absorbing compound aids in improving animal health. Toxins, and in particular mycotoxins or aflatoxins are produced by molds that can be present in crop, like fusarium or aspergillus species. It appears that certain clay exhibit a good absorption of both aflatoxins and other non polar mycotoxins, which is an advantage. The clay used in the present invention preferably exhibits absorption of about 30% or more of vomitoxine. In another embodiment of the invention, the clay used exhibits absorption of about 40% or more of zearelanone. The absorption is measured with in vitro absorption and in-vivo validation techniques (M. Sabater—Veterniary Faculty University Utrecht, Sala de Miguel—Laboratorios Atheleias Buenos Aires).

The clay is preferably a bentonite and or/and montmorillonite type clay, preferably containing ileite and ambrosite type silicates. The clay product may have free flowing and pelleting aid properties besides mycotoxin binding.

Formulations with Fatty Acids or Other Organic Acids

In one preferred embodiment, the butyrate salt is combined with other organic acids with 3 carbon atoms or more up to 40 carbon atoms. Suitable organic acids with 3 carbon atoms or more include middle chain-length fatty acids, lactic acid, and long chain highly unsaturated fatty acids and the derivatives thereof. The organic acids may be used as esters with mono or polyfunctional alcohols, as esters of metals or the like; generally it is less preferred to use these acids as free acid. In case the compounds are liquid at a temperature below 40° C., it is preferred to use these for example absorbed on a carrier material as it is preferred to use solid components in the formulation. A suitable carrier may be clay, silica or the like.

Suitable middle chain length fatty acids are fatty acids with 5-14 carbon atoms such as caproic acid, caprylic acid or capric acid and their salts. Included are also medium chain triglycerides (MCT's), diglycerides and monoglycerides and mixtures of these.

Preferred long chain highly unsaturated acids include fatty acids with 16 to 30 carbon atoms and at least 2 carbon-carbon double bonds. Suitable examples include linoleic acid; oleic acid, arachidonic acid (ARA); eicosatrenoic acid, eicosapentaenoic acid (EPA), docosapentaenoic acid and docosahexaenoic acid (DHA).

Suitable amounts are amounts as described above.

The combined effects of butyric acid and specific medium chain fatty acids or glyceride-esters may in particular improve feed efficiency because of healthier animals.

The combined effects of butyric acid and highly unsaturated fatty acids are in particular increased growth of the animals and nutritional quality of the animal products.

For the combination with lactic acid or derivative there-of below a more specific example is described.

Formulation with Lactic Acid or Derivate (Salt)

In one preferred embodiment of the invention, a feed additive for poultry comprises a butyrate salt component, a lactate salt component, and a compound absorbing toxins.

Butyrate salts can be used in combination with lactate salt as feed additive. This combination appeared to show a synergistic effect on reducing bacterial infections and improving growth performance, feed efficiency, feed intake, quality of the animal products. Lactate is preferably used as calcium salt, as such salt is stable, and provides further calcium to the animal.

The feed additive for poultry further comprises a toxin absorbing clay as described above.

The feed additive for poultry preferably contains about 50 g or more of butyrate salt, about 50 g or more of lactate salt and about 50 g or more of a toxin absorbing compound, to be added to a ton of feed. It is preferred to formulate the feed additive such that one kg preferably is added to one ton of feed. In such case, it is preferred to have about 10% or more of any of the three components per kg of feed additive. Generally, any of the three components will be present in amount of 70% or less. Preferably, respective amounts are about 20% or more of each compound, and about 50% or less of each compound. In formulating such feed additive, it is preferred to first determine the amount of butyrate salt needed, and thereafter determine a suitable amount of lactate salt. The toxin binder can be used to make 1 kg; preferably, about 100 g per kg of toxin binder or more is used.

### Prebiotica, Probiotica and Cell Extracts

In a further embodiment, the butyric acid salt is combined with prebiotic compounds. These are compounds that cause the gastrointestinal tract to develop more, and healthier microorganisms. Suitable prebiotica include, but are nor limited to oligosaccharides, such as for example fructo-, manno-, or galactooligosaccharides. The combined effect of butyric acid salt and prebiotica is a further improvement of the health of the animal, further reducing the necessity to use medical treatment such as antibiotics.

In a further embodiment, the butyrate acid salt is combined with probiotic and/or cell extracts. Probiotics may be living cells, like lactobacillus, bacillus, enterococcus, or saccharomyces, which aid in healthy intestine flora. Also extracts of these species may be useful, like cell-membrane, cell wall and the like. Preferably, the bacillus or coccus species are species are obtained from the gastrointestinal tract of the animal species.

### Fat Soluble Vitamins

In a further embodiment, the butyrate salt is combined with fat soluble vitamins like vitamin A, B, D, and/or E. In stead of the vitamin itself, it is also possible to use metabolites of such vitamins. A particularly preferred vitamin is vitamin D3 and its metabolite 25-hydroxy and the 1.25-hydroxy (cholecalciferol).

### Animal Feed

The butyrate salt, preferably in combination with other active ingredients is mixed with base feed for animals. The feed additive preferably is used in about 1 kg per ton of feed, as this is easy to put into practice. However, other mixing amounts may be useful as well.

The standard feed may comprise cereals like carbohydrate source, like corn wheat and barley, sorghum, rice and their by-products, cassava; protein sources like soybean meal, rapeseed meal, sunflower, whey powder, milk powder, fish meal and the like.

The feed additive of the present invention may be in the form of pellets, mash or crumble, and can be used as mixed with complete feed, or in premixes.

The feed with the feed additive of the present invention is useful for a wide variety of animals. Suitable animals include swine and piglets, calves and cattle, all type of poultry, sheep and other farm animals like rabbits and aquaculture species.

The present invention does not relate to, and excludes, the use of butyrate salts and formulations comprising butyrate salts in combination with other active compounds for human or (human) pharmaceutical use.

The invention will be further elucidated with examples, without being limited thereto

### EXAMPLES 1-3

Calciumbutyrate was prepared from 100 kg butyric acid (food grade), and 50/50 molar amount of CaO and Ca(OH)₂. Water was condensed and treated in a waste-water facility. The crystalline mixture obtained was milled and screened.

Calciumbutyrate granules were made in a rotogranulation machine. The hard fraction of coconut and palm oil was injected into a batch of calciumbutyrate. The first one or two injection caused the butyrate crystals to form granules. The further injections of the fats caused a coating to be formed, with about 2-5 wt % of CaBut. The granules were sieved, and the fraction of 0.2 to 1.0 mm was obtained for feed additive purpose. The larger granules were milled, and—together with the smaller fraction—was used to make a further grade of CaBut granules. The first type of granules contained 84% of CaBut (70% butyric), the second batch contained 72% CaBut (60% butyric). Both grades were nearly smell free, the second grade performed even better.

In a comparable way, sodiumbutyrate salt was prepared and granulated. In order to obtain a stable, non-hygroscopic smell free product, it was useful to add 20 wt % of calciumcarbonate, and to use 50-60 wt % of fats, such that a product with 20-30% of NaBut was obtained.

### EXAMPLES 4-10

With the granular CaBut, the following mixtures can be made, showing improved health benefits and/or quality of food product.

| | Example | | | | | | |
|---|---|---|---|---|---|---|---|
| | 4 | 5 | 6 | 7 | 8 | 9 | 10 |
| Product | g | g | g | g | g | g | g |
| CaBut 70% granules | 320 | | | 400 | | 300 | |
| CaBut 60% granules | | 380 | | | 300 | | |
| NaBut 30% granules | | | 700 | | | | 700 |
| CaLactate (95% crystalline) | 380 | 320 | | | | | |
| Bentonite clay | 300 | 300 | 200 | | | | |
| Thymol (35% on clay) | | | | | | 300 | |
| Tannin extract (80%) | | | | 400 | | | |
| Bacillus probiotic | | | | | 300 | | |
| Capric acid triglyceride | | | | | | 400 | |
| (absorbed on silica) | | | | | | | |
| Fish oil (40%) (absorbed on clay) | | | | | | | 300 |
| Gallactose oligomers | | | 100 | | 400 | | |
| Calciumcarbonate | | | | 100 | | | |
| 25-hydroxy vitamin D3 | | | | 100 | | | |

The products of example 4 and 5 show, if fed to chicken in 0.5 to 1.5 kg per ton of normal feed, increased egg shell strength and feed conversion. The product of example 6 shows an improvement in health and resistance against bacterial intrusion in the gastrointestinal tract. Even better results are obtained in comparison with example 6, with the formulation of example 7. Examples 8 and 9 are further optimized in this respect. The mixture of example 10 shows improved growth in piglets.

1. Formulation for use in animal feed comprising coated granules comprising butyrate salt, the formulation further comprising one or more other active ingredients.

2. The formulation according to claim 1, wherein the butyrate salt is a sodium or calcium salt, and preferably a calcium salt.

3. Formulation according to claim 1, wherein the granules have a particle size of 0.1 mm or more, preferably 0.2 mm or more.

4. Formulation according to claim 1, wherein the granules have a particle size be about 2 mm or less, and preferably about 1 mm or less.

5. Formulation according to claim 1, wherein the granules comprise a binder and a coating.

6. Formulation according to claim 1, wherein the granule is stable at about 50° C. or higher, preferably up to about 55° C. or higher.

7. Formulation according to claim 1, wherein the granule comprises fats as binder and as coating, preferably glyceride esters of fatty acids, alkyl esters of fatty acids, fatty acids or mixtures thereof.

8. Formulation according to claim 1, wherein the granules contain about 20 wt % butyrate salt or more, preferably about 50 wt % or more.

9. Formulation according to claim 1, wherein the active ingredient is chosen from the group consisting of, plant extracts, prebiotic compounds, probiotics, yeast extracts, short chain fatty acid(s), middle-chain length fatty acids, unsaturated long chain fatty acids, lactate salt or derivate, fat soluble vitamin and toxin absorbing compound.

10. Formulation according to claim 1, wherein two active ingredients are chosen from the group of claim 9.

11. Formulation according to claim 10, comprising a butyrate salt component, a lactate salt component, and a compound absorbing toxins for use as additive in poultry feed.

12. Formulation according to claim 9, wherein the active ingredient is a plant extract, and preferably tannin extract of the sweet chestnut tree is used.

13. Formulation according to claim 9, wherein each of the active ingredients is used in amounts of about 50 g or more per ton of feed, preferably about 100 g or more per ton of feed and wherein the amount is about 10 kg per ton of feed or less, preferably about 5 kg per ton or less.

14. Formulation according to claim 1, wherein the coated granules contain the butyrate salt as sole active compound.

15. Use of a formulation according to claim 1 as feed additive for animal feed.

16. Feed for animals comprising a feed and a formulation according to claim 1.
